(19) United States
(12) Patent Application Publication
(10) Pub. No.: US 2025/0340871 A1
(43) Pub. Date: Nov. 6, 2025

(54) COMPOSITION FOR CONTROLLING DNA DAMAGE REPAIR EFFICIENCY

(71) Applicant: Industry-Academic Cooperation Foundation, Yonsei University, Seoul (KR)

(72) Inventors: Hyun Woo PARK, Seoul (KR); Dong Hyeon KIM, Seoul (KR)

(73) Assignee: Industry-Academic Cooperation Foundation, Yonsei University, Seoul (KR)

(21) Appl. No.: 18/697,159

(22) PCT Filed: Sep. 27, 2022
(86) PCT No.: PCT/KR2022/014428

§ 371 Date: Nov. 8, 2024

(30) Foreign Application Priority Data
Oct. 1, 2021 (KR) 10-2021-0130531
Mar. 3, 2022 (KR) 10-2022-0027681

(51) Int. Cl. C12N 15/113

(52) U.S. Cl. CPC C12N 15/113; C12N 2310/14; C12N 2310/20; C12N 2310/531

(57) ABSTRACT

The present invention relates to a composition for promoting homologous recombination (HR) and a composition for preventing or treating homologous recombination-deficient disease comprising the same. The present invention may be efficiently used to activate homologous recombination for the treatment of diseases, including tumors, by inhibiting the formation of TEAD-complex2, a new complex which is formed by the binding between the N-terminal region of the TEAD protein and DNA damage response-related proteins. Accordingly, the present invention may not only be used to treat various homologous recombination-deficient diseases, including tumors, but also may dramatically improve gene editing efficiency in genetic scissor technology such as the CRISPR/Cas system by promoting homologous recombination after DNA double-strand breakage.

### TECHNICAL FIELD

This application claims the benefit of the filing dates of Korean Patent Application No. 10-2021-0130531, filed with the Korean Intellectual Property Office on Oct. 1, 2021, and of Korean Patent Application No. 10-2022-0027681, filed with the Korean Intellectual Property Office on Mar. 3, 2022, the entire contents of which are incorporated herein.

The present invention relates to a method of promoting or inhibiting DNA damage repair by regulating the binding between TEAD protein and a series of proteins.

### BACKGROUND ART

DNA damage is a stress that inevitably occurs during cell division and survival, and about 200 or more proteins are directly or indirectly involved in 1) recognition of DNA damage, 2) signal transduction for DNA damage, and 3) repair of DNA damage. Defects in DNA damage repair mechanisms lead to genomic instability, causing various diseases, including tumors. In addition, defects in DNA damage repair mechanisms lead to a decrease in immune function and the development of related diseases as the DNA damage repair mechanisms are essential for immune cell activation and antibody production. Therefore, identifying various types of DNA damage, repair mechanisms, and proteins involved therein, and identifying their specific functions are very important issues in understanding molecular biological mechanisms and establishing therapeutic strategies for diseases through these mechanisms.

Transcription factors are proteins that bind to DNA and regulate the expression of specific genes. Research has been conducted on which transcription factors are involved in the expression of which genes and to identify signaling pathways through which the expression is regulated. However, little research has been conducted on other functions of transcription factors other than their inherent function of transcribing specific genes and regulating the expression thereof.

Accordingly, the present inventors have sought to identify specific transcription factors involved directly in DNA damage recognition and repair mechanisms in addition to transcriptional regulation in the cell nucleus and regulate their activity, thereby proposing anticancer therapeutic strategies based on inhibition of DNA damage repair mechanisms, therapeutic strategies for diseases caused by defects in DNA damage repair mechanisms, and strategies for improving gene editing efficiency by increasing homologous recombination efficiency.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made extensive research efforts to discover effective molecular targets that can efficiently activate or inhibit DNA damage repair mechanisms. As a result, the present inventors have identified for the first time that TEAD protein, previously known only as a transcription factor of the Hippo pathway that binds to YAP/TAZ, binds directly to proteins involved in DNA damage response and repair, and have found that the efficiency of DNA homologous recombination is reduced by this binding, and that when the expression of activity of the TEAD protein is inhibited or the interaction between the TEAD proteins and the YAP/TAZ proteins is inhibited, the efficiency of homologous recombination is significantly increased, thereby completing the present invention.

Therefore, an object of the present invention is to provide a composition for promoting homologous recombination (HR) and a composition for preventing or treating homologous recombination-deficient disease comprising the same.

Another object of the present invention is to provide a composition for promoting non-homologous end joining (NHEJ).

Still another object of the present invention is to provide a method for screening a composition for promoting homologous recombination (HR).

Other objects and advantages of the present invention will be more apparent from the following detailed description, the appended claims, and the accompanying drawings.

### Technical Solution

According to one aspect of the present invention, the present invention provides a composition for preventing or treating homologous recombination-deficient disease comprising an inhibitor of TEA domain (TEAD) protein as an active ingredient.

The present inventors have made extensive research efforts to discover effective molecular targets that can efficiently activate or inhibit DNA damage repair mechanisms. As a result, the present inventors have identified for the first time that TEAD protein, previously known only as a transcription factor of the Hippo pathway that binds to YAP/TAZ, binds directly to proteins involved in DNA damage response and repair, and have found that the efficiency of DNA homologous recombination is reduced by this binding, and that when the expression or activity of the TEAD protein is inhibited or the interaction between the TEAD proteins and the YAP/TAZ proteins is inhibited, the efficiency of homologous recombination is significantly increased.

### Advantageous Effects

The features and advantages of the present invention are summarized as follows:
(a) The present invention provides a composition for promoting homologous recombination (HR) and a composition for preventing or treating homologous recombination-deficient disease comprising the same.
(b) The present invention may be efficiently used to activate homologous recombination for the treatment of diseases, including tumors, by inhibiting the formation of TEAD-complex2, a new complex which is formed by the binding between the TEAD protein, more specifically, the N-terminal region of the TEAD protein, and DNA damage response-related proteins.
(c) The present invention may not only be used to treat various homologous recombination-deficient diseases, including tumors, but also may dramatically improve gene editing efficiency in genetic scissor technology such as the CRISPR/Cas system by promoting homologous recombination after DNA double-strand breakage.

### BRIEF DESCRIPTION OF DRAWINGS

FIGS. 1a and 1b show screening results for transcription factors that participate in the DNA damage repair mechanism or bind to proteins involved in the DNA damage repair mechanism. FIG. 1a shows the results of selecting transcription factors GATA1, SPIB, BTG2, IKZF1, and NFE2, which are co-localized with damaged DNA markers, by immunofluorescence assay. FIG. 1b shows the results of immunoprecipitation indicating that GATA1 binds to PARP, p53 binds to RPA1/2, and YAP binds to RPA1/2 and PARP.

FIGS. 2a to 2d show that TEAD has an independent function other than transcriptional function by binding to YAP/TAZ. FIG. 2a shows that cell death by the inhibition of YAP/TAZ-TEAD was not observed in K562 and H146 cells without YAP/TAZ or in OCM1 and HT29 cells in which YAP/TAZ is always inactivated. FIG. 2b shows the results of Western blot analysis indicating the decrease in TEAD expression in each cell type. FIG. 2c shows that the inhibition of cell growth by inhibition of TEAD was not observed in cells in which YAP/TAZ are inhibited or absent. FIG. 2d shows that the binding of TEAD to a promoter for transcription decreased in the absence of YAP/TAZ.

FIGS. 3a to 3j show that the transcription factor TEAD binds to proteins involved in the DNA damage repair mechanism, competitively with YAP/TAZ. FIG. 3a shows the results of investigating the binding between TEAD and Rad51 by Bioplex interactome. FIG. 3b shows the results of confirming the binding of TEAD4 to PARP, Ku80, LIG3, RFC1, Ku70, and XRCC1 by mass spectrometry. FIG. 3c shows the results of confirming the independent binding of TEAD and RPA2 to YAP/TAZ at the cellular level. FIG. 3d shows the results of confirming the YAP/TAZ-independent binding of TEAD and Rad51 at the cellular level. FIG. 3e shows that TEAD and proteins involved in the DNA damage repair mechanism are inhibited by YAP/TAZ at the cellular level. FIG. 3f shows that the N-terminus of TEAD is essential for the binding between Rad51 and TEAD. FIG. 3g shows results indicating that the binding between Rad51 and TEAD is inhibited by YAP. FIG. 3h shows the results of confirming the binding of TEAD 1/2/4 to Ku70 at the cellular level. The proteins that bind to the N-terminus of TEAD were newly named complex2, and to distinguish therefrom, YAP/TAZ was named complex1. FIG. 3i schematically shows the configurations of complex1 and complex2. In FIG. 3j, sequences in TEAD1/2/3/4 essential for the formation of complex2 are schematically represented.

FIGS. 4a to 4g show results indicating that TEAD mitigates DNA replication fork stress, causing fork collapse. FIG. 4a shows that, in the absence of TEAD, damage caused by DNA replication stress was significantly less. FIG. 4b shows that single-stranded DNA exposure increased in the absence of TEAD. FIG. 4c shows that, in the absence of TEAD, phosphorylation of RPA by HU was restored by inhibition of ATR. FIG. 4d shows that, in the absence of TEAD, DNA replication stress caused by HU was less, and FIG. 4e shows that, in the absence of TEAD, the cell viability depending on HU increased. This phenomenon was observed in all of cells (Molm14) without YAP/TAZ, cells (OCM1) in which YAP/TAZ are inhibited, and cells (211H) in which YAP/TAZ are activated, as shown in FIG. 4f, and cells (H146) without YAP/TAZ as shown in FIG. 4g.

FIGS. 5a to 5f show that TEAD promotes non-homologous end joining (NHEJ) by inhibiting BRCA1 and activating 53BP1. FIG. 5a shows that 53BP1 activates NHEJ in the presence of TEAD and activates BRCA1 in the absence of TEAD in order to repair CPT-induced DNA double-strand breaks. FIG. 5b shows that Rad51, which is essential for HR, is activated in the absence of TEAD. FIG. 5c shows the results of Western blot analysis indicating that phosphorylation of RPA, an HR signal, was maintained for a long time in the absence of TEAD when repairing DNA double-strand breaks. FIG. 5d confirms that the viability against DNA double strand breaks increases in the absence of TEAD. FIG. 5e shows the construction of an EJ5-GFP reporter system for measuring the efficiency of NHEJ and an HR reporter system for measuring the efficiency of HR, and shows that when TEAD was silenced or overexpressed, NHEJ decreased and HR increased, or NHEJ increased and HR decreased. FIG. 5f shows that HR-mediated knock-in efficiency increased in the absence of TEAD, as measured by an mClover reporter system capable of measuring gene editing efficiency.

### BEST MODE

In the present specification, the term “homologous recombination-deficient disease” is meant to encompass any pathological condition in which homologous recombination, which, upon the occurrence of DNA double strand breaks (DSBs), repairs the damaged strand using an undamaged homologous DNA strand as a template, is inactivated or does not proceed normally so that cytotoxicity caused by DNA damage cannot be eliminated. Failure of DSB repair results in fatal consequences such as genomic instability and cell death, and inappropriate end joining due to DSB repair failure is a major cause of oncogenic transformation due to chromosomal translocation.

According to a specific embodiment of the present invention, the homologous recombination-deficient disease that can be prevented or treated by the composition of the present invention is a homologous recombination-deficient tumor.

More specifically, the homologous recombination-deficient tumor is selected from the group consisting of homologous recombination-deficient breast cancer, ovarian cancer, peritoneal cancer, lymphoma, glioblastoma multiforme, gliosarcoma, astrocytoma, glioblastoma, medulloblastoma, glioma, supratentorial primitive neuroectodermal tumor, atypical teratoid/rhabdoid tumor, choroid plexus carcinoma, malignant ganglioma, cerebral gliomatosis, meningioma, and paraganglioma.

In the present specification, the term “inhibitor of TEA domain (TEAD) protein” refers not only to a substance that causes a decrease in the activity or expression of TEAD protein so that the activity or expression of TEAD becomes undetectable or TEAD exists at an insignificant level, but also to a substance that decreases the activity or expression of TEAD to the extent that homologous recombination inhibited by the binding between TEAD and proteins such as PARP, Ku80/70, RFC1, Rad51, and RPA1/2 can be significantly improved.

In the present specification, the term “decrease in expression” may mean a state in which the expression level of TEAD decreased by, for example, at least 20%, more specifically at least 30%, even more specifically at least 40%, compared to that in a control group.

In the present specification, the term “decrease in activity” refers to a measurable and significant decrease in the unique in vivo function of TEAD compared to that in a control group. Specifically, the term “decrease in activity” refers to a decrease in the activity of TEAD to the extent that homologous recombination inhibited by binding to a DNA damage response-related protein in a subject can be significantly improved or restored. The decrease in activity includes not only a simple decrease in function but also the ultimate inhibition of activity due to a decrease in stability.

According to a specific embodiment of the present invention, the TEAD protein is selected from the group consisting of TEAD1, TEAD2, TEAD3 and TEAD4.

According to a specific embodiment of the present invention, the inhibitor of TEAD protein is an antibody or an antigen-binding fragment thereof that specifically binds to a polypeptide comprising the amino acid sequence of at least one of SEQ ID NOS: 1 to 4, or an aptamer that specifically binds to a polypeptide comprising the amino acid sequence of at least one of SEQ ID NOs: 1 to 4.

According to the present invention, the amino acid sequence of SEQ ID NO: 1 corresponds to residues 30 to 101 at the N-terminus of TEAD1 protein, the amino acid sequence of SEQ ID NO: 2 corresponds to residues 40 to 111 at the N-terminus of TEAD2 protein, the amino acid sequence of SEQ ID NO: 3 corresponds to residues 30 to 101 at the N-terminus of TEAD3 protein, and the amino acid sequence of SEQ ID NO: 4 corresponds to residues 1 to 75 at the N-terminus of TEAD4 protein.

The present inventors have found for the first time that, apart from the fact that the C-terminal region of TEAD protein exerts a unique function as a transcription factor that regulates the expression of genes essential for cancer survival and division by binding to YAP/TAZ, the N-terminus of TEAD protein binds to proteins directly involved in DNA damage response and repair, competitively with YAP/TAZ, thereby inhibiting their binding to single strands of DNA at double strand break (DSB) sites, thus reducing the efficiency of homologous recombination. Therefore, the antibody or aptamer that specifically binds to the amino acid sequence of any one of SEQ ID NOS: 1 to 4 is able to block the homologous recombination inhibitory effect of the N-terminal region of TEAD protein.

The antibody that specifically recognizes the TEAD protein is a polyclonal or monoclonal antibody, and is preferably a monoclonal antibody.

The antibody of the present invention may be produced by methods commonly practiced in the art, for example, the fusion method (Kohler and Milstein, European Journal of Immunology, 6:511-519 (1976)), the recombinant DNA method (U.S. Pat. No. 4,816,567), or the phage antibody library method (Clackson et al, Nature, 352:624-628 (1991) and Marks et al, J. Mol. Biol., 222:58, 1-597 (1991)). General procedures for antibody production are described in detail in Harlow, E. and Lane, D., Using Antibodies: A Laboratory Manual, Cold Spring Harbor Press, New York, 1999; and Zola, H., Monoclonal Antibodies: A Manual of Techniques, CRC Press, Inc., Boca Raton, Florida, 1984.

In the present specification, the term “antigen-binding fragment” refers to a portion of a polypeptide among the entire structure of an immunoglobulin, to which an antigen may bind. Examples of the antigen-binding fragment include, but are not limited to, F(ab′)2, Fab′, Fab, Fv, and SCFV.

In the present specification, the term “specifically binding” has the same meaning as “specifically recognizing”, and means that an antigen and an antibody (or a fragment thereof) specifically interact with each other through an immunological reaction.

According to the present invention, it is also possible to inhibit the activity of TEAD protein using an aptamer, which specifically binds to the TEAD protein, instead of the antibody. In the present specification, the term “aptamer” refers to a single-stranded nucleic acid (RNA or DNA) molecule or peptide molecule that binds to a specific target substance with high affinity and specificity. General contents of aptamers are disclosed in detail in Hoppe-Seyler F, Butz K “Peptide aptamers: powerful new tools for molecular medicine”. J Mol Med. 78(8):426-30(2000); Cohen B A, Colas P, Brent R. “An artificial cell-cycle inhibitor isolated from a combinatorial library”. Proc Natl Acad Sci USA. 95(24):14272-14277(1998).

According to a specific embodiment of the present invention, the inhibitor of TEAD protein is a nucleic acid molecule that inhibits the expression of a polynucleotide encoding a polypeptide comprising the amino acid sequence of at least one of SEQ ID NOs: 1 to 4.

In the present specification, the term “nucleic acid molecule” is meant to encompass DNA (gDNA and cDNA) and RNA molecules. Nucleotides, which are the basic structural units in nucleic acid molecules, include not only natural nucleotides, but also analogues having modified sugar or base moieties (Scheit, Nucleotide Analogs, John Wiley, New York (1980); Uhlman and Peyman, Chemical Reviews, 90:543-584 (1990)).

In the present specification, the term “nucleic acid molecule that inhibits expression” refers to a nucleic acid molecule comprising a complementary nucleic acid sequence capable of hybridizing to a target gene, which is capable of specifically recognizing the target gene and causing a modification in the nucleotide structure, which causes deterioration of the function of the target gene. Examples of the nucleic acid molecule include shRNA, siRNA, miRNA, ribozyme, PNA, antisense oligonucleotides, and gRNA included in the CRISPR system.

In the present specification, the term “complementary” means that the nucleic acid molecule for inhibiting expression is sufficiently complementary to a target nucleic acid sequence so as to hybridize selectively to the target nucleic acid sequence under certain annealing or hybridization and is meant to include both substantially complementary and perfectly complementary, and preferably refers to perfectly complementary. In the present specification, the term “substantially complementary sequence” is meant to include not only a perfectly matched sequence, but also a sequence that is partially mismatched with the sequence to be compared, to the extent that sequence-specific hybridization can occur by annealing to a specific sequence.

In the present specification, the term “shRNA (small hairpin RNA)” is a single-stranded RNA sequence consisting of 50-70 nucleotides, which forms a stem-loop structure in vivo and has a tight hairpin structure for silencing the target gene expression via RNA interference. Typically, complementary long RNAs of 19-29 nucleotides on both sides nucleotides are base-paired of a loop portion of 5-10 together to form a double-stranded stem. shRNA is transduced into cells through a vector containing a U6 promoter for constitutive expression and is usually passed on to daughter cells so that silencing of the target gene is inherited.

In the present specification, the term “siRNA” refers to a short double-stranded RNA capable of inducing RNA interference (RNAi) phenomenon by cleavage of a specific mRNA. It consists of a sense RNA strand having a sequence homologous to the mRNA of the target gene and an antisense RNA strand having a sequence complementary thereto. The total length thereof may be 10 to 100 bases, preferably 15 to 80 bases, most preferably 20 to 70 bases, and the terminal structure thereof may be either blunt or cohesive as long as it is capable of inhibiting expression of the target gene by the RNAi effect. The cohesive terminal structure may be both a 3′-terminal protrusion structure and a 5′-terminal protrusion structure.

In the present specification, the term “miRNA (microRNA)” is an oligonucleotide that is not expressed in cells, and refers to a single-stranded RNA molecule, which has a short stem-loop structure and inhibits expression of the target gene by complementary binding to the mRNA of the target gene.

In the present specification, the term “ribozyme” refers to a type of RNA molecule that functions to recognize and cleave the nucleotide sequence of a specific RNA, like an enzyme. The ribozyme is a nucleotide sequence complementary to the target mRNA strand and consists of a region that binds to target mRNA with specificity and a region that cleaves the target RNA.

In the present specification, the term “PNA (peptide nucleic acid)” refers to a molecule having the characteristics of both nucleic acid and protein, which is capable of complementarily binding to DNA or RNA. PNA is not found in nature but is artificially synthesized by chemical methods, and it regulates the expression of the target gene by forming a double strand through hybridization with a natural nucleic acid having a complementary nucleotide sequence.

In the present specification, the term “antisense oligonucleotide” is a nucleotide sequence complementary to the sequence of a specific mRNA, and refers to a nucleic acid molecule that binds to a complementary sequence in the target mRNA and inhibits essential activities for translation of the target mRNA into protein, translocation into the cytoplasm, maturation, or other overall biological functions. The antisense oligonucleotide may be modified at one or more base, sugar or backbone positions to enhance efficacy (De Mesmaeker et al., Curr Opin Struct Biol., 5(3):343-55, 1995). The oligonucleotide backbone may be modified with phosphorothioate, phosphotriester, methyl phosphonate, short-chain alkyl, cycloalkyl, short-chain heteroatomic, heterocyclic sugar sulfonate, or the like.

In the present specification, the term “guide RNA (gRNA)” refers to an RNA molecule that is used in a gene editing system that recognizes a target gene and induces a nuclease to specifically cleave the recognized site. Typical examples of this gene editing system include a CRISPR (Clustered Regularly Interspaced Short Palindromic Repeats) system.

The above-described nucleic acid molecule of the present invention is able to inhibit the expression of TEAD protein at the gene level by, for example, its expression in a subject with homologous recombination-deficient disease.

In the present specification, the term “expressing” or “expression” means allowing a subject to express an exogenous gene or artificially introducing an endogenous gene using a gene delivery system to increase the natural expression level of the endogenous gene, thereby making the introduced gene replicable as an extrachromosomal factor or by chromosomal integration in a subject's cell. Accordingly, the term “expression” is synonymous with “transformation”, “transfection” or “transduction”.

As used herein, the term “gene delivery system” refers to any means for delivering a gene into a cell, and the term “gene delivery” has the same meaning as intracellular transduction of a gene. At the tissue level, the term “gene delivery” has the same meaning as the spread of a gene. Accordingly, the gene delivery system of the present invention may be referred to as a gene transduction system or a gene spread system.

More specifically, the nucleic acid molecule specifically binds to the nucleotide sequence of at least one of SEQ ID NOS: 5 to 8.

According to the present invention, the nucleotide sequence of SEQ ID NO: 5 is a nucleotide sequence encoding the amino acid sequence of SEQ ID NO: 1, the nucleotide sequence of SEQ ID NO: 6 is a nucleotide sequence encoding the amino acid sequence of SEQ ID NO: 2, the nucleotide sequence of SEQ ID NO: 7 is a nucleotide sequence encoding the amino acid sequence of SEQ ID NO: 3, and the nucleotide sequence of SEQ ID NO: 8 is a nucleotide sequence encoding the amino acid sequence of SEQ ID NO: 4.

In the present invention, it is obvious to those skilled in the art that the nucleotide sequences whose expression is to be inhibited are not limited to the nucleotide sequences shown in the attached sequence listing.

Some variations in nucleotides do not result in variations in proteins. Such nucleic acids include all nucleic acid molecules having functionally equivalent codons, codons encoding the same amino acid (e.g., due to codon degeneracy, six codons for arginine or serine), or codons encoding biologically equivalent amino acids.

Considering the above-described variations having biological equivalent activity, the nucleotide sequence of the present invention is construed to also include sequences having substantial identity to the sequence set forth in the sequence listing. The “substantial identity” refers to a sequence having at least 60%, preferably at least 70%, more preferably at least 80%, most specifically 90% homology, when aligning the above-described sequence of the present invention with any other sequence to maximally correspond to each other and analyzing the aligned sequence using an algorithm commonly used in the art.

Alignment methods for sequence comparison are known in the art. Various methods and algorithms for alignment are disclosed in Higgins and Sharp, CABIOS 5:151-3 (1989); Corpet et al., Nuc. Acids Res. 16:10881-90 (1988); Huang et al., Comp. Appl. BioSci. 8:155-65 (1992), and Pearson et al., Meth. Mol. Biol. 24:307-31 (1994). NCBI Basic Local Alignment Search Tool (BLAST) (Altschul et al., J. Mol. Biol. 215:403-10 (1990)) is available from the National Center for Biological Information (NCBI), etc., and may be used on the Internet in connection with sequence analysis programs, such as blastp, blastm, blastx, tblastn, and tblastx.

In the present specification, the term “prevention” means inhibiting the occurrence of a disorder or a disease in a subject who has never been diagnosed as having the disorder or disease, but is likely to suffer from such a disorder or disease.

In the present specification, the term “treatment” means (a) inhibiting the progress of a disorder, disease or symptom; (b) alleviating the disorder, disease or symptom; or (c) eliminating the disorder, disease or symptom. When the composition of the present invention is administered to a subject, it functions to inhibit the progress of symptoms caused by homologous recombination deficiency, or to eliminate or alleviate symptoms, by blocking the homologous recombination inhibitory effect of the N-terminal region of TEAD protein by inhibiting the activity or expression of the TEAD protein. Thus, the composition of the present invention may serve as a therapeutic composition for the disease alone, or may be administered in combination with other pharmacological ingredients and applied as a therapeutic aid for the disease. Accordingly, as herein used, the term “treatment” or “therapeutic agent” encompasses “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition containing a pharmacological ingredient sufficient to provide a therapeutic or prophylactic effect to a subject to whom/which the pharmaceutical composition of the present invention is to be administered. Accordingly, the term “therapeutically effective amount” is meant to include a “prophylactically effective amount”.

In the present specification, the term “subject” includes, without limitation, humans, mice, rats, guinea pigs, dogs, cats, horses, cows, pigs, monkeys, chimpanzees, baboons or rhesus monkeys. Specifically, the subject of the present invention is a human.

When the composition of the present invention is prepared as a pharmaceutical composition, the pharmaceutical composition of the present invention comprises a pharmaceutically acceptable carrier.

Examples of the pharmaceutically acceptable carrier that is comprised in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, which are commonly used in formulation. The pharmaceutical composition of the present invention may further comprise a lubricant, a wetting agent, a sweetening agent, a flavoring agent, an emulsifying agent, a suspending agent, a preservative, and the like, in addition to the above-described ingredients. Suitable pharmaceutically acceptable carriers and agents are described in detail in Remington's Pharmaceutical Sciences (19th ed., 1995).

The pharmaceutical composition of the present invention may be administered orally or parenterally. Specifically, it may be administered orally, intravenously, subcutaneously, or intraperitoneally.

An appropriate dosage of the pharmaceutical composition of the present invention may vary depending on various factors such as formulation method, administration mode, patient's age, weight, sex, pathological condition, diet, administration time, administration route, excretion rate, and reaction sensitivity. A preferred dosage of the pharmaceutical composition of the present invention is within the range of 0.001 to 100 mg/kg for an adult.

The pharmaceutical composition of the present invention may be prepared in a unit dose form or prepared to be contained in a multi-dose container by formulating with a and/or excipient, pharmaceutically acceptable carrier according to a method that may be easily carried out by a person skilled in the art. In this case, the formulation of the pharmaceutical composition may be a solution, suspension, syrup or emulsion in oil or aqueous medium, or an extract, powder, granule, tablet or capsule, and may further comprise a dispersing agent or a stabilizer.

The details mentioned with respect to the composition and pharmaceutical composition of the present invention apply equally unless they contradict each other.

According to another aspect of the present invention, provided is a method for preventing or treating homologous recombination-deficient disease, comprising a step of administering to a subject in need thereof an inhibitor of TEA domain (TEAD) protein.

According to still another aspect of the present invention, provided is the use of an inhibitor of TEA domain (TEAD) protein for the manufacture of a medicament for use in the prevention or treatment of homologous recombination-deficient disease.

In the above-described method or use, the above detailed description of the composition and the pharmaceutical composition may be applied.

According to yet another aspect of the present invention, the present invention provides a composition for promoting homologous recombination (HR) comprising an inhibitor of TEA domain (TEAD) protein as an active ingredient.

Since inhibitor of TEAD protein used in the present invention and the homologous recombination promoting effect thereof have already been described in detail, the description thereof will be omitted to avoid excessive overlapping. The composition of the present invention inhibits the binding between TEAD protein, specifically the N-terminal region of TEAD protein, and DNA damage response-related proteins, allowing these proteins to bind well to the single strand of DNA at DSB sites. The homologous recombination promoted thereby may not only be used to treat various homologous recombination-deficient diseases, but also can dramatically improve the knock-in efficiency in genetic editing technologies involving a DSB step, such as zinc finger, TALEN, and CRISPR/Cas systems.

According to another aspect of the present invention, the present invention provides a composition for promoting non-homologous end joining (NHEJ) comprising TEA domain (TEAD) protein or a functional fragment thereof as an active ingredient.

As shown in the examples described below, the present inventors have identified for the first time that the N-terminal region of TEAD protein binds to PARP and Ku70/80, thereby promoting non-homologous end joining (NHEJ) in which non-complementary ends are joined together when DNA double-strand breaks (DSBs) occur. Typically, cells selectively utilize two major DSB repair mechanisms: homologous recombination (HR) and non-homologous end joining (NHEJ). When the number of protein fragments in TEAD protein or a functional portion thereof, specifically the N-terminal region thereof that binds to PARP and Ku70/80, increases, the DNA double-strand break repair mechanism may be selectively induced by inhibiting homologous recombination and increasing non-homologous end joining.

According to a specific embodiment of the present invention, the functional fragment of the TEAD protein used in the present invention comprises the amino acid sequence of at least one of SEQ ID NOs: 1 to 4.

According to another aspect of the present invention, the present invention provides a method for screening a composition for promoting homologous recombination (HR), the method comprising steps of:
(a) bringing a candidate substance into contact with a biological sample containing a TEA domain (TEAD) protein or cells expressing the same; and
(b) measuring the activity or expression level of the TEAD protein in the sample,
wherein, if the activity or expression level of the TEAD protein decreased, the candidate substance is determined as the composition for promoting homologous recombination.

Since the TEAD protein used in the present invention and the composition for promoting homologous recombination using the same have already been described in detail, the description thereof will be omitted to avoid excessive overlapping.

In the present invention, the term “biological sample” refers to any sample containing TEAD protein or cells expressing the same, obtained from mammals, including humans, and includes, but is not limited to, tissues, organs, cells, or cell cultures.

The term “candidate substance” used while referring to the screening method of the present invention refers to an unknown substance that is added to a sample containing TEAD protein or cells expressing the same and is used in screening to examine whether or not it affects the activity or expression level of TEAD. Examples of the test substance include, but are not limited to, compounds, nucleotides, peptides, and natural extracts. The step of measuring the expression level or activity of TEAD in the biological sample treated with the test substance may be performed by various expression level and activity measurement methods known in the art. If the measurement result shows that the expression level or activity of TEAD decreased, the test substance may be determined as a composition for promoting homologous recombination of DNA.

According to the present invention, the expression level of TEAD protein, which is a screening target of the present invention, may be measured according to an immunoassay method using an antigen-antibody reaction. This immunoassay may be performed according to various immunoassay or immunostaining protocols developed previously.

For example, when the method of the present invention is performed according to a radioimmunoassay method, antibodies labeled with radioisotopes (e.g., ¹⁴C, ¹²⁵I, ³²P and ³⁵S) may be used.

According another embodiment of the present invention, the expression level of TEAD protein, which is a screening target of the present invention, may be measured at the gene level using an agent for measuring the expression level of the gene encoding the TEAD protein. The agent for measuring the expression level of the gene is, for example, a primer or probe that specifically binds to the nucleic acid sequence of the TEAD gene.

As used in the present specification, the term “primer” refers to an oligonucleotide which serves as a starting point for synthesis under conditions in which synthesis of a primer extension product complementary to a nucleic acid chain (template) is induced, i.e., in the presence of nucleotides and a polymerization agent such as DNA polymerase and at a suitable temperature and pH. Specifically, the primer is a deoxyribonucleotide single chain. The primers that are used in the present invention may include naturally occurring dNMPs (i.e., dAMP, dGMP, dCMP and dTMP), modified nucleotides or non-natural nucleotides. In addition, the primers may also include ribonucleotides.

In the present specification, the term “probe” refers to natural or modified monomers, including deoxyribonucleotides and ribonucleotides capable of hybridizing to a specific nucleotide sequence, or linear oligomers having linkages. Specifically, the probe is single-stranded for maximum efficiency in hybridization. More specifically, the probe is a deoxyribonucleotide. As the probe that is used in the present invention, a sequence perfectly complementary to a specific nucleotide sequence of the TEAD gene may be used, but a sequence substantially complementary thereto may also be used as long as it does not interfere with specific hybridization. In general, the stability of a duplex formed by hybridization tends to be determined by the match of sequences at the ends, and thus it is preferred to use a probe which is complementary to the 3′-end or 5′-end of the target sequence.

Suitable conditions for hybridization may be determined by referring to Joseph Sambrook, et al., Molecular Cloning, A Laboratory Manual, Cold Spring Harbor Laboratory Press, N.Y. (2001) and Haymes, B. D., et al., Nucleic Acid Hybridization, A Practical Approach, IRL Press, Washington, D.C. (1985).

According to another aspect of the present invention, the present invention provides a method for screening a composition for promoting homologous recombination (HR), the method comprising steps:
(a) bringing a candidate substance into contact with a biological sample containing TEA domain (TEAD) protein or cells expressing the same, and at least protein selected from the group consisting of PARP, Ku80, Ku70, LIG3, RFC1, Rad51, RPA1, RPA2, and XRCC1, or cells expressing the same; and
(b) measuring the binding between the TEAD protein and the at least one protein selected from the group consisting of RFC1, PARP, Ku80, Ku70, LIG3, RFC1, Rad51, RPA1, RPA2, and XRCC1, in the sample,
wherein, if the binding decreased, the candidate substance is determined as the composition for promoting homologous recombination.

Since the TEAD protein used in the present invention, the composition for promoting homologous recombination, and the biological sample used for screening have already been described in detail, the description thereof will be omitted to avoid excessive overlapping.

In the present specification, the term “decrease in binding” means that the binding between the TEAD protein, specifically the N-terminal region thereof, and at least one protein selected from the group consisting of RFC1, PARP, Ku80, Ku70, LIG3, RFC1, Rad51, RPA1, RPA2, and XRCC1, is inhibited so that the binding between these DNA damage response-related proteins and the single strand of DNA at double strand break (DSB) sites is restored to a measurable level. This decrease in n binding may be achieved by competitive binding of the candidate substance to the TEAD protein to interfere with the interaction between the TEAD protein and the above-listed DNA damage response-related proteins, or may be achieved by decreasing the activity or expression of the TEAD protein. Specifically, the decrease in binding may mean a state in which binding decreased by at least 20%, more specifically at least 40%, even more specifically at least 60%, compared to that in a control group.

### MODE FOR INVENTION

Hereinafter, the present invention will be described in more detail by way of examples. These examples are only for explaining the present invention in more detail, and it will be apparent to those skilled in the art that the scope of the present invention according to the subject matter of the present invention is not limited by these examples.

### EXAMPLES

### Experimental Methods

### Cell Culture

H293A cells were cultured in DMEM supplemented with 10% FBS and 1% penicillin/streptomycin and maintained in a 5% CO₂ incubator at 37° C.

### Production of TEAD1/2/4 Knock-Out (KO) Cells

shRNA and gRNAs for TEAD1/2/4 were used to inhibit the expression of the TEAD gene in H293A cells. Each gRNA was designed with reference to the CRISPR design tool (http://crispr.mit.edu), and the shRNA and gRNA nucleotide sequences are shown in Table 1 below. shRNA and gRNAs were cloned into pSpCas9(BB)-2A-Puro (PX459) (Addgene no. 48139) which was then transduced into H293A cells. After 24 hours, TEAD1/2/4 KO cells were selected and maintained for 2 to 3 days using puromycin.

TABLE 1

| | Nucleotide | |
|---|---|---|
| | sequence | |
| Name | (5′→3′) | SEQ ID NO. |
| shTEAD1/2/4 | ATGATCAACTT | SEQ ID NO: 9 |
| | CATCCACAAG | |
| gRNA for TEAD1 | TGGCAGTGGCC | SEQ ID NO: 10 |
| | GAGACGATC | |
| gRNA for TEAD2 | AGATAGGTGGG | SEQ ID NO: 11 |
| | ACGCCGGCG | |
| gRNA for TEAD4 | TCAAGGATCTC | SEQ ID NO: 12 |
| | TTCGAACG | |

### Immunoprecipitation (IP)

For IP, Myc-tagged TEAD was transduced into H293A cells which were then cultured for 24 hours. The cells were lysed on ice with 0.5% NP-40 lysis buffer supplemented with a protease phosphatase inhibitor cocktail. The lysed cells were pipetted 2 to 30 times every 10 minutes to loosen the pellet. Next, the cells were lysed on ice for 30 minutes, and then centrifuged at 13,000 rpm for 10 minutes at 4° C., and the supernatant was collected. Myc-conjugated magnetic beads were added to the supernatant which was then incubated on a rotator at 4° C. for 18 hours. Proteins bound to the beads were collected with a magnetic bar and washed four times with 0.5% NP-40 lysis buffer, and then immunoprecipitated proteins were eluted with laemmli buffer.

### Immunofluorescence Assay

For immunofluorescence assay, cells were cultured on glass coverslips. After washing once with PBS, the cells were fixed with 4% paraformaldehyde for 15 minutes at room temperature. After washing three times with PBS, the cells were permeabilized with 0.2% (v/v) Triton X-100 for 10 minutes and washed three times with PBS for 10 minutes. The cells were blocked with 3% BSA for 30 minutes and incubated with primary antibodies (Rad51 #8875 Cell Signaling Technology, 53BP1 #88439 Cell Signaling Technology, RPA32 ab2175 abcam, RPA70 ab97338 abcam, RPA32 S33 A300-246A Bethyl, H2A.X (Ser139) 05-636 Merck, Phospho RPA32 (S4/S8) A300-245A Bethyl, BRCA1 sc-6954 Santa Cruz Biotechnology, TEAD4 SC-101184 Santa Cruz Biotechnology) in a shaker at 4° C. for 18 hours. After washing again with PBS three times for 10 minutes, the cells were incubated with secondary antibody diluted in 3% BSA in the dark for 2 hours. After washing with PBS three times for 10 minutes, the cells were stained with DAPI solution and washed twice with PBS. After mounting with Prolong antifade reagent, images were acquired using a LEICA DMI800 confocal microscope.

### DR-GFP/NHEJ Assay

To produce H293A DR-GFP cells, a linearized pDR-GFP (Addgene #26475) plasmid was transformed and inserted into the genomic DNA of cells. To produce H293A EJ5-GFP cells, a linearized pimEJ5GFP (Addgene #44026) plasmid was inserted into the genomic DNA of cells using the same method. For each cell type, cells were selected and maintained using puromycin (4 μg/ml). To measure the efficiency of DNA double-strand damage repair, the cells were transformed with the I-SecI plasmid and cultured for 48 to 72 hours, and then the percentage of GFP+ cells was measured by flow cytometry.

mClover Assay

To measure gene targeting efficiency, H293A WT, and H293A TEAD1/2/4 KO cells were co-transformed with the donor plasmid and gRNA plasmid and cultured for 72 hours, and then the percentage of GFP+ cells was measured by flow cytometry.

### Cell Viability Assay

H293A WT and H293A TEAD 1/2/4 KO cells were seeded in 24-well plates at a density of 100,000 cells per well. After 24 hours, the cells were treated with CPT (0.01, 0.03, 0.1, 0.3, and 1 μM) and hydroxyurea (0.01, 0.03, 0.1, 0.3, and 1 mM) for 48 hours, and then treated with MTT reagent for 2 hours. After dissolving in DMSO, the absorbance at 540 nm was measured.

### Experiment Results

### Identification of Transcription Factors Involved in DNA Damage Repair Mechanism

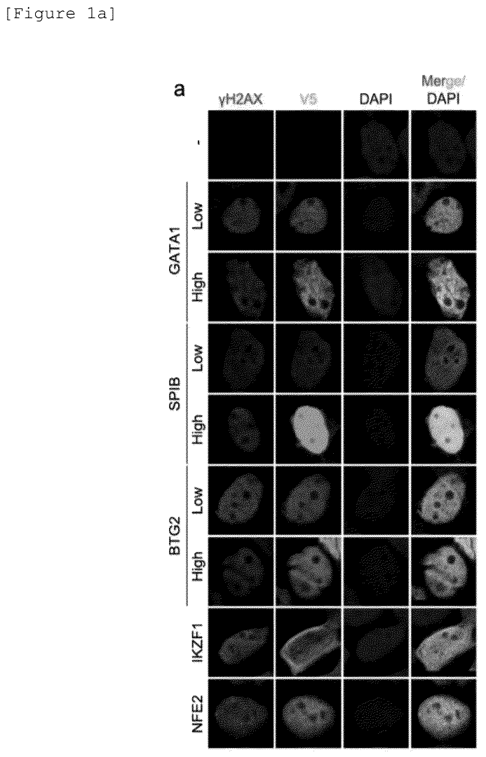

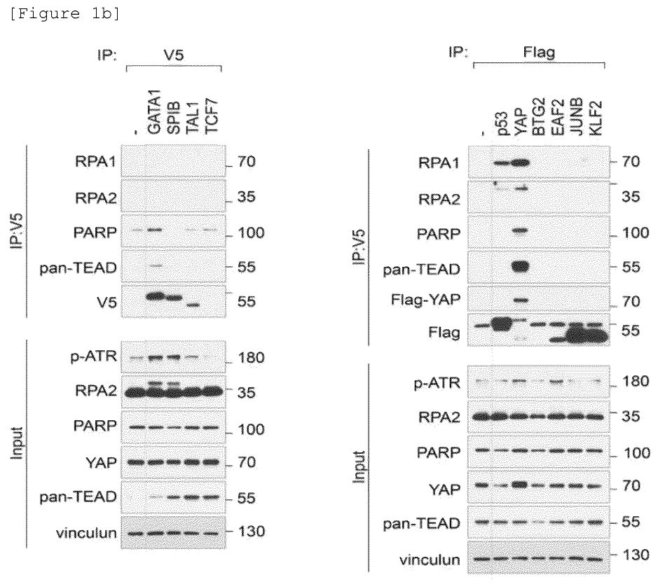

As a result of identifying proteins involved in the DNA damage repair mechanism or transcription factors that bind to these proteins, the transcription factors GATA1, SPIB, BTG2, IKZF1 and NFE2 co-localized with the damaged DNA marker were selected by immunofluorescence assay (FIG. 1a). In addition, it was confirmed through immunoprecipitation that GATA1 bound to PARP, p53 bound to RPA1/2, and YAP bound to RPA1/2 and PARP (FIG. 1b).

TEAD Performs Independent Function Other than Transcriptional Function by Binding to YAP/TAZ.

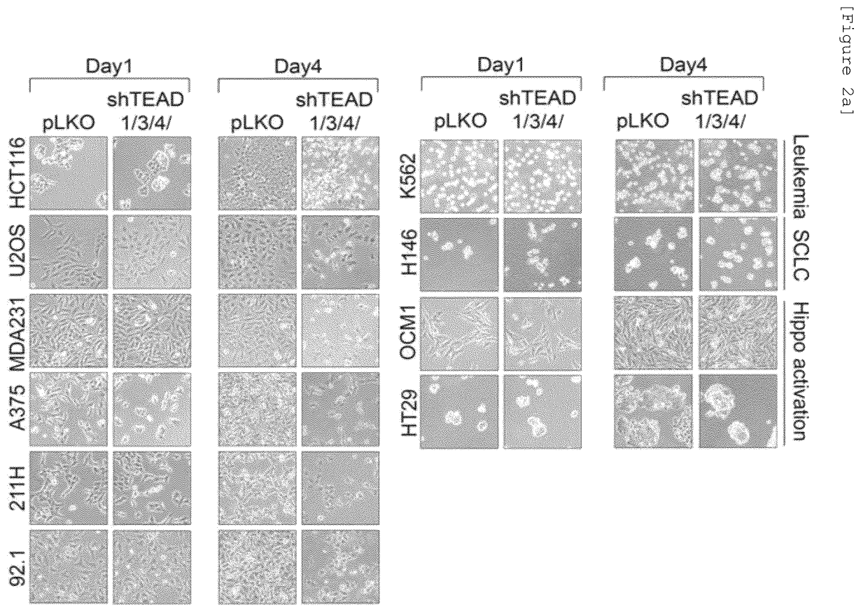

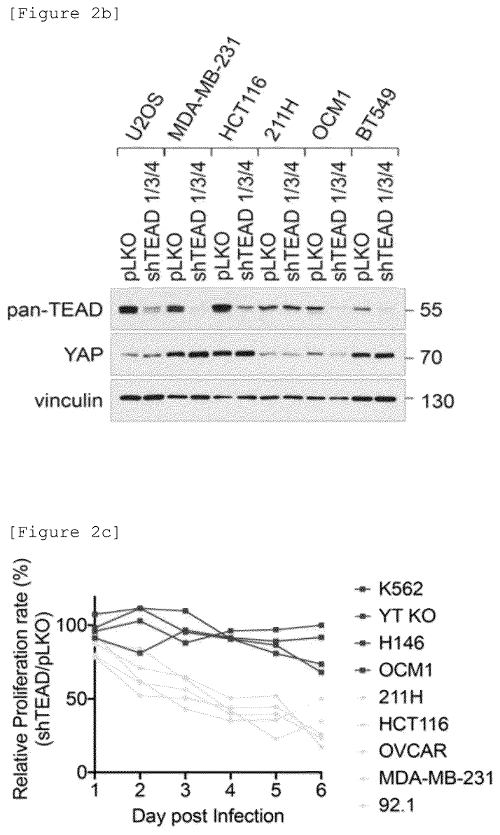

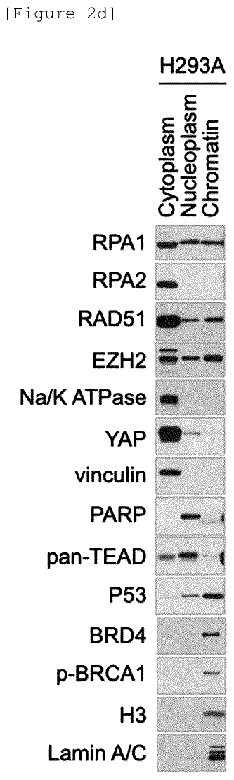

In K562 and H146 cells without YAP/TAZ, and in OCM1 and HT29 cells in which YAP/TAZ are always inactivated, cell death was not observed even when YAP/TAZ-TEAD was inhibited (FIG. 2a). It was confirmed that through Western blot analysis that TEAD expression was decreased in each cell type (FIG. 2b). In cells where YAP/TAZ were inhibited or not expressed, the inhibition of cell growth by TEAD inhibition was not observed (FIG. 2c). In addition, it was shown that TEAD existed in the nucleoplasm region independently of YAP within the cells, suggesting that it can function independently as a transcription factor (FIG. 2d).

TEAD Binds to Proteins Involved in DNA Damage Repair Mechanism Competitively with YAP/TAZ

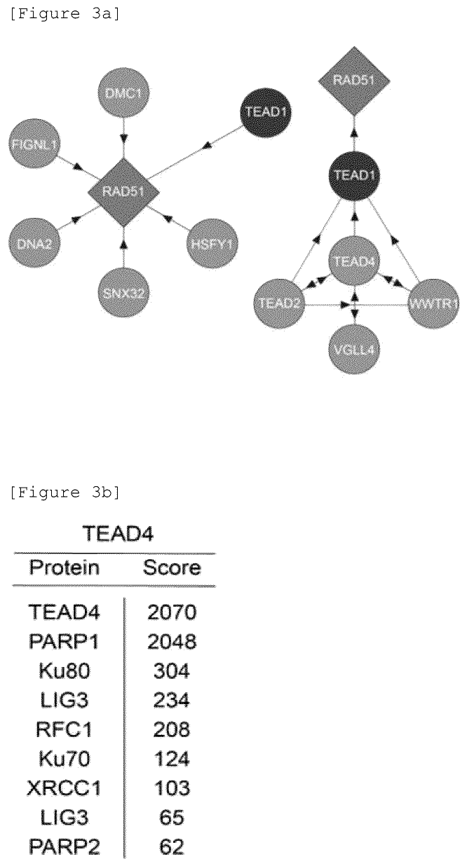

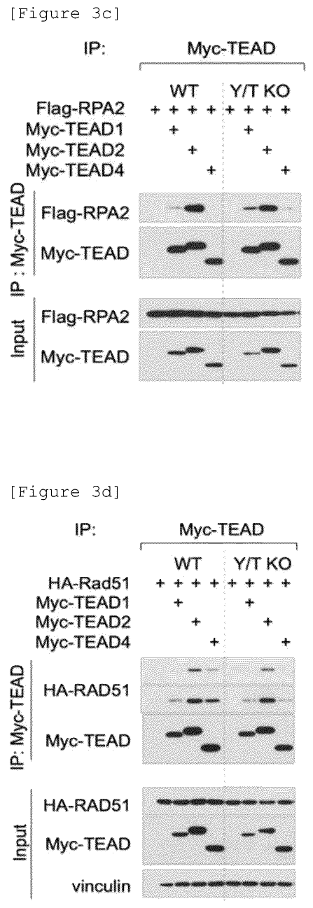

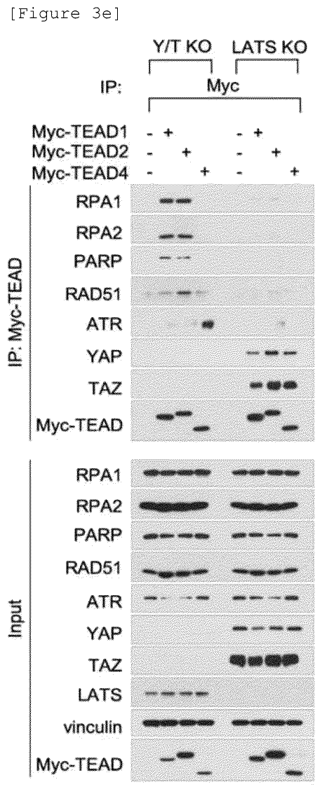

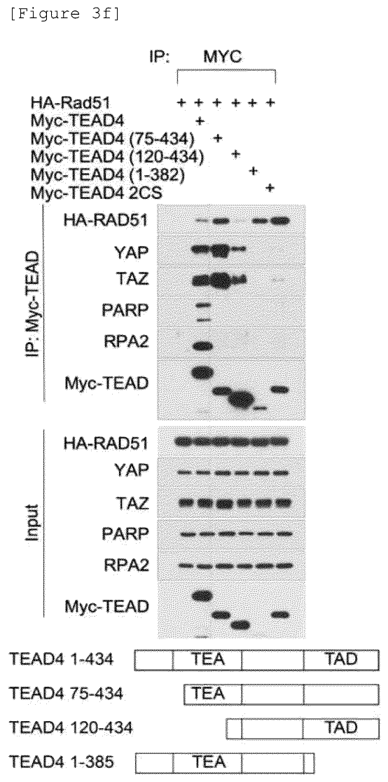

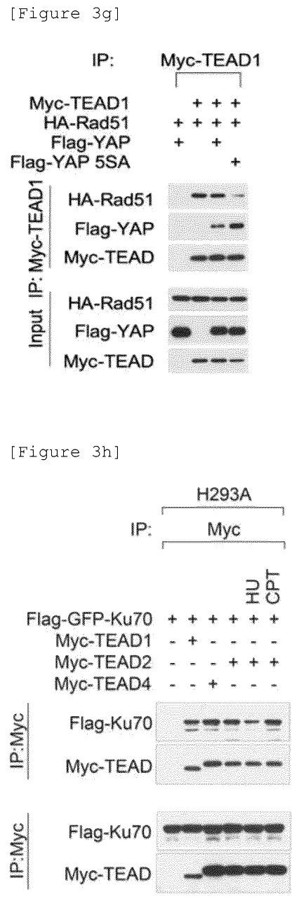

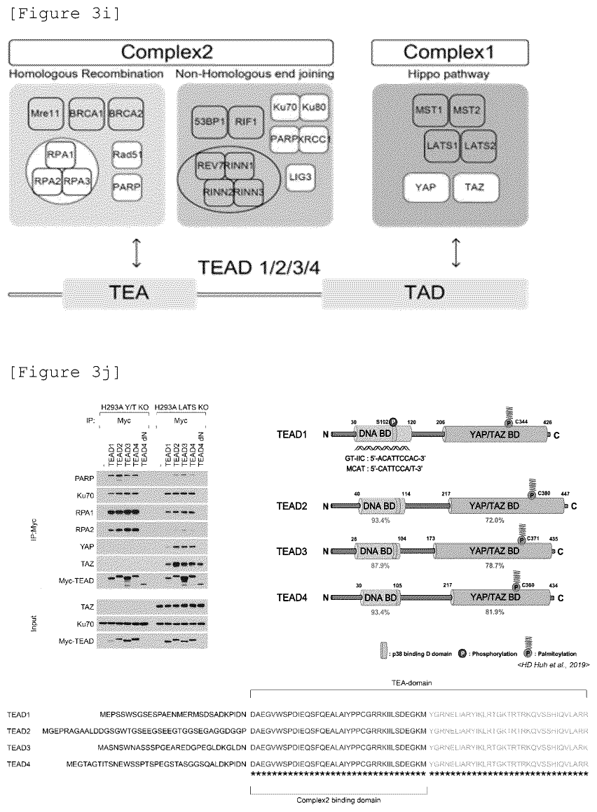

It was confirmed through Bioplex interactome that TEAD binds to Rad51 (FIG. 3a). The results of mass spectrometry showed that TEAD4 bound to PARP, Ku80, LIG3, RFC1, Ku70, and XRCC1 (FIG. 3b). In addition, it was confirmed at the cellular level that TEAD and RPA2 bound to each other independently of YAP/TAZ (FIG. 3c), and that TEAD and Rad51 also bound to each other independently of YAP/TAZ (FIG. 3d). It was confirmed at the cellular level that TEAD and proteins involved in the DNA damage repair mechanism were inhibited by YAP/TAZ (FIG. 3e), and it could be confirmed that the N-terminus of TEAD was essential for the binding between Rad51 and TEAD (FIG. 3f). Meanwhile, the binding between Rad51 and TEAD was inhibited by YAP (FIG. 3g), and TEAD 1/2/4 and Ku70 bound together (FIG. 3h).

As described above, in the present invention, the proteins that bind to the N-terminus of TEAD were newly named complex2, and to distinguish therefrom, YAP/TAZ were named complex1 (FIG. 3i).

### TEAD Mitigates DNA Replication Fork Stress, Causing Fork Collapse

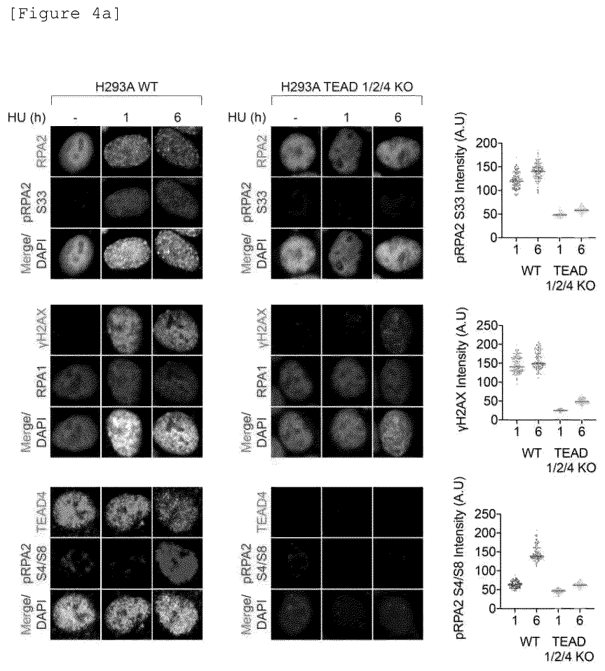

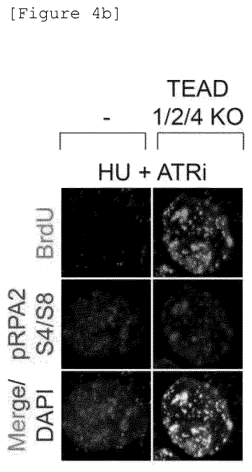

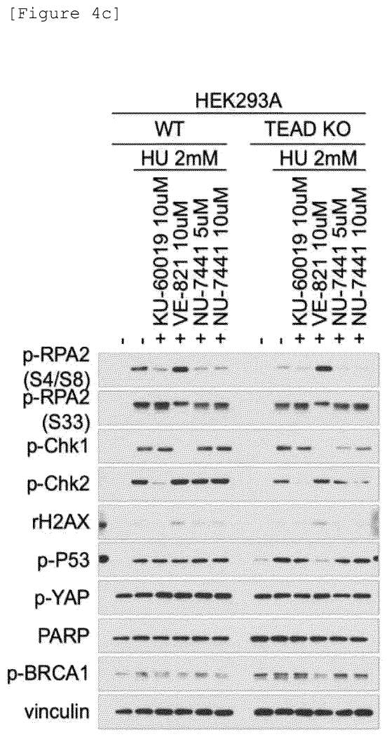

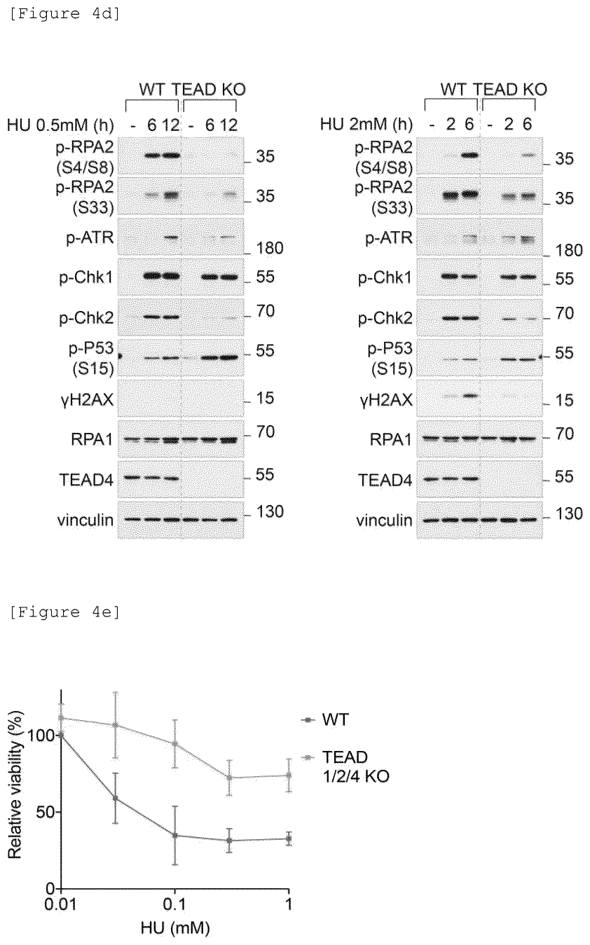

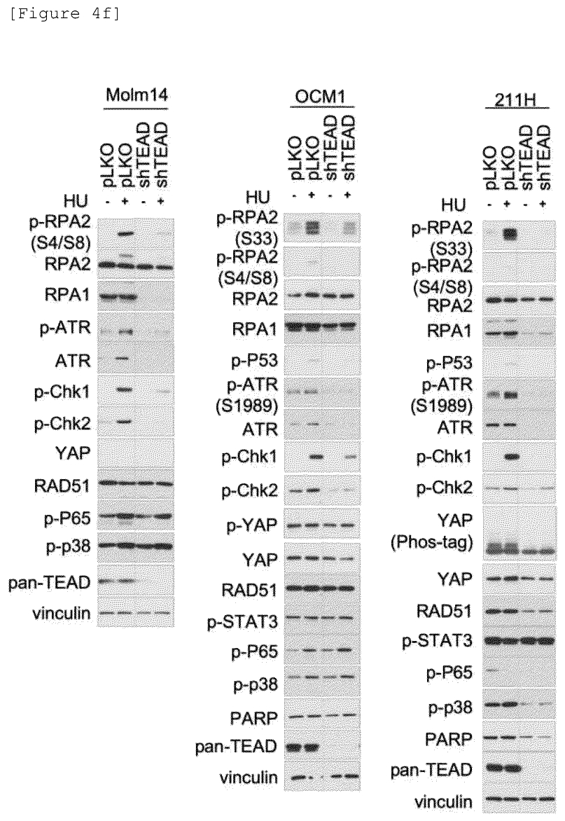

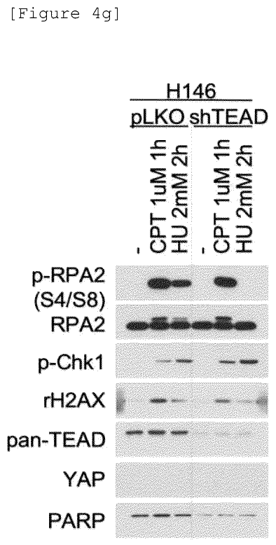

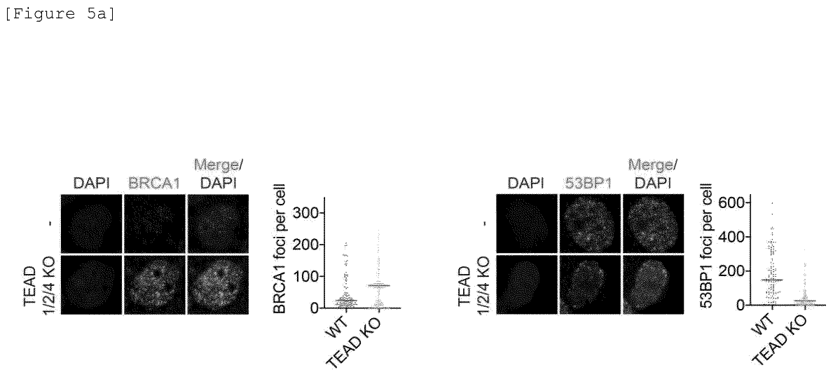

Damage caused by DNA replication stress was significantly reduced in the absence of TEAD (FIG. 4a), and single-stranded DNA exposure was increased in the absence of TEAD (FIG. 4b). In the absence of TEAD, phosphorylation of RPA by HU was restored by inhibition of ATR (FIG. 4c). In the absence of TEAD, DNA replication stress caused by HU was less (FIG. 4d), and in the absence of TEAD, the cell viability depending on HU increased (FIG. 4e). This phenomenon was observed in all of cells (Molm14) without YAP/TAZ, cells (OCM1) in which YAP/TAZ are inhibited, cells (211H) in which YAP/TAZ are activated, and cells (H146) without YAP/TAZ (FIGS. 4f and 4g).

### TEAD Promotes Non-Homologous End Joining (NHEJ) by Inhibiting BRCA1 and Activating 53BP1

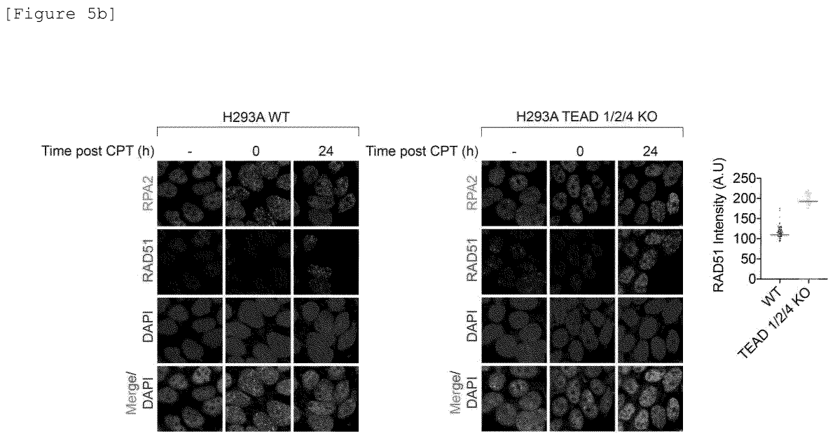

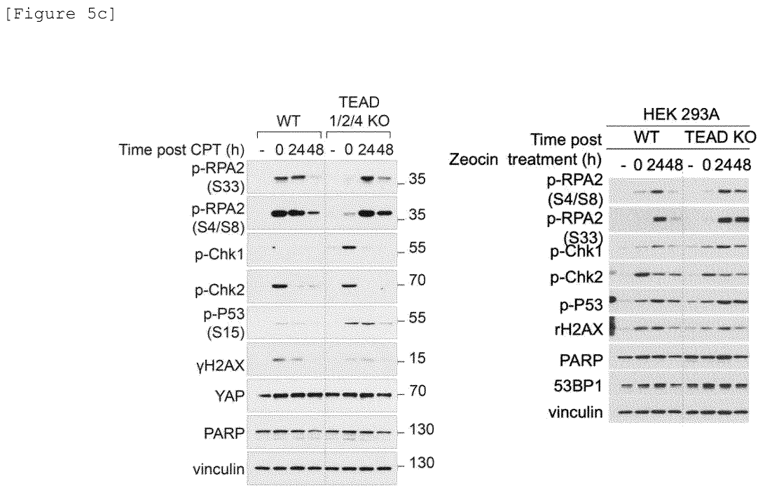

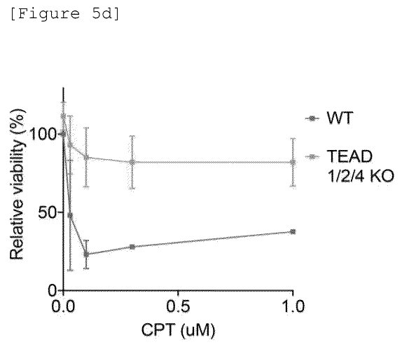

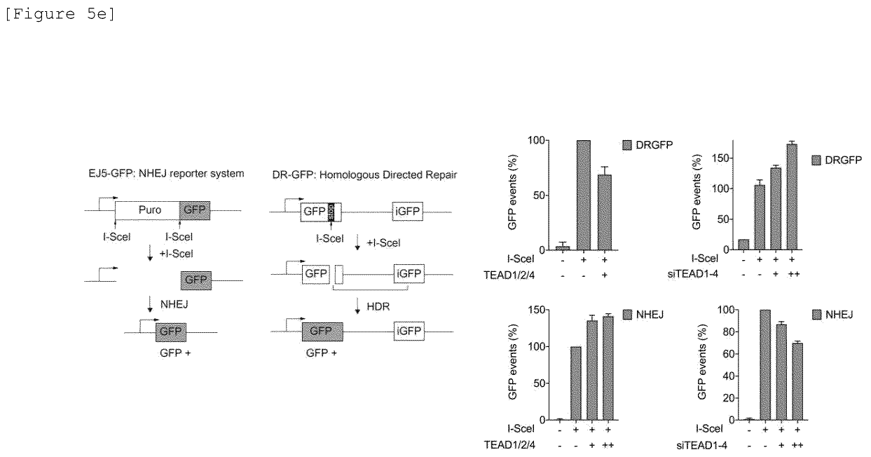

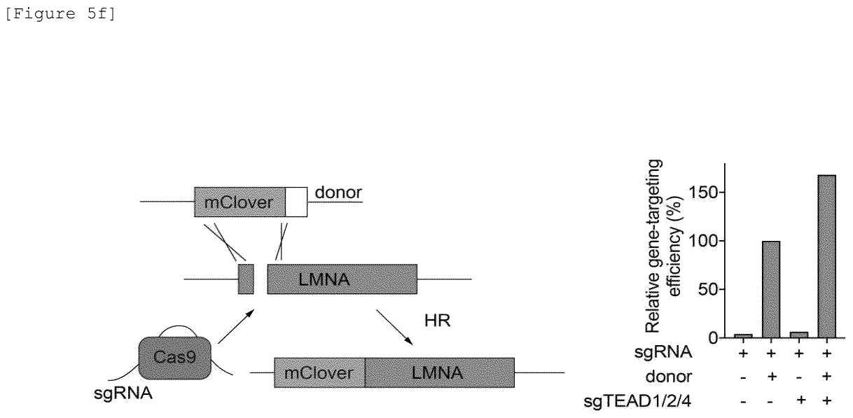

It was confirmed that 53BP1 activated NHEJ in the presence of TEAD and activated BRCA1 in the absence of TEAD in order to repair CPT-induced DNA double-strand breaks (FIG. 5a). It was confirmed that Rad51, which is essential for HR, was activated in the absence of TEAD (FIG. 5b). It was confirmed through Western blot analysis that phosphorylation of RPA, an HR signal, was maintained for a long time in the absence of TEAD when repairing DNA double-strand breaks (FIG. 5c). It was confirmed that, in the absence of TEAD, the viability increased even when DNA double strand breaks occurred (FIG. 5d). Through the constructed EJ5-GFP reporter system for measuring the efficiency of NHEJ and the constructed HR reporter system for measuring the efficiency of HR, it was confirmed that when TEAD was silenced or overexpressed, NHEJ decreased and HR increased, or NHEJ increased and HR decreased (FIG. 5e). Through the mClover reporter system capable of measuring gene editing efficiency, it could be seen that HR-mediated knock-in efficiency increased in the absence of TEAD (FIG. 5f).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1. A method for preventing or treating homologous recombination-deficient disease comprising administering an inhibitor of TEA domain (TEAD) protein in a pharmaceutically effective amount to a subject in need thereof.

2. The method of claim 1, wherein the TEAD protein is selected from the group consisting of TEAD1, TEAD2, TEAD3, and TEAD4.

3. The method of claim 1, wherein the inhibitor of TEAD protein is an antibody or an antigen-binding fragment thereof that specifically binds to a polypeptide comprising the amino acid sequence of at least one of SEQ ID Nos: 1 to 4, or an aptamer that specifically binds to a polypeptide comprising the amino acid sequence of at least one of SEQ ID Nos: 1 to 4.

4. The method of claim 1, wherein the inhibitor of TEAD protein is a nucleic acid molecule that inhibits expression of a polynucleotide encoding a polypeptide comprising the amino acid sequence of at least one of SEQ ID NOs: 1 to 4.

5. The method of claim 1, wherein the homologous recombination-deficient disease is selected from the group consisting of a homologous recombination-deficient tumor, Fanconi anemia, Bloom syndrome, and ataxia telangiectasia.

6. The method of claim 5, wherein the homologous recombination-deficient tumor is selected from the group consisting of homologous recombination-deficient breast cancer, ovarian cancer, peritoneal cancer, lymphoma, glioblastoma multiforme, gliosarcoma, astrocytoma, glioblastoma, medulloblastoma, glioma, supratentorial primitive neuroectodermal tumor, atypical teratoid/rhabdoid tumor, choroid plexus carcinoma, malignant ganglioma, cerebral gliomatosis, meningioma, and paraganglioma.

7. A method for promoting homologous recombination (HR) and inhibiting non-homologous end joining (NHEJ) comprising administering an inhibitor of TEA domain (TEAD) protein in pharmaceutically effective amount to a subject in need thereof.

8. A method for inhibiting homologous recombination (HR) and promoting non-homologous end joining (NHEJ) comprising administering TEA domain (TEAD) protein or a functional fragment thereof in a pharmaceutically effective amount to a subject in need thereof.

9. The method of claim 8, wherein the functional fragment of the TEAD protein comprises the amino acid sequence of at least one of SEQ ID NOs: 1 to 4.

10. (canceled)

11. (canceled)

12. (canceled)

13. (canceled)


SEQUENCE LISTING (12 sequences)

SEQ ID NO: 1 - protein, 72 aa, synthetic construct
DAEGVWSPDIEQSFQEALAIYPPCGRRKIILSDEGKMYGRNELIARYIKLRTGKTRTRKQVSSHIQVLARRK

SEQ ID NO: 2 - protein, 72 aa, synthetic construct; identical to SEQ ID NO: 1

SEQ ID NO: 3 - protein, 72 aa, synthetic construct
DAEGVWSPDIEQSFQEALAIYPPCGRRKIILSDEGKMYGRNELIARYIKLRTGKTRTRKQVSSHIQVLARKK

SEQ ID NO: 4 - protein, 75 aa, synthetic construct
MEGTAGTITSNEWSSPTSPEGSTASGGSQALDKPIDNDAEGVWSPDIEQSFQEALAIYPPCGRRKIILSDEGKMY

SEQ ID NO: 5 - DNA, 216 bp, synthetic construct
  source 1..216: mol_type = other DNA
gatgcagaaggggtctggagccccgacatcgagcaaagctttcaggaggccctggctatctatccaccatgtgggaggaggaaaatcatcttatcagacgaaggcaaaatgtatggtaggaatgaattgatagccagatacatcaaactcaggacaggcaagacgaggaccagaaaacaggtgtctagtcacattcaggttcttgccagaaggaaa

SEQ ID NO: 6 - DNA, 216 bp, synthetic construct
  source 1..216: mol_type = other DNA
gatgcagagggggtgtggagcccagacattgagcagagcttccaggaggccctggccatctatccaccctgcggccgccggaaaataattttgtctgatgaaggcaagatgtatggtcggaatgaactgatcgcccgctacatcaagctgagaacggggaagacccgaactcgaaaacaggtttctagtcacatccaggttttggcccgaaggaaa

SEQ ID NO: 7 - DNA, 216 bp, synthetic construct
  source 1..216: mol_type = other DNA
gatgcggagggcgtgtggagcccggacatcgagcagagcttccaggaggccctggccatctacccgccctgcggccggcggaagatcatcctgtcagacgagggcaagatgtacggccgaaatgagttgattgcacgctatattaaactgaggacggggaagactcggacgagaaaacaggtgtccagccacatacaggttctagctcggaagaag

SEQ ID NO: 8 - DNA, 225 bp, synthetic construct
  source 1..225: mol_type = other DNA
ttggagggcacggccggcaccattacctccaacgagtggagctctcccacctcccctgaggggagcaccgcctctgggggcagtcaggcactggacaagcccatcgacaatgacgcagagggcgtgtggagcccggatattgagcagagtttccaggaggccctcgccatctacccgccctgtggcaggcgcaaaatcatcctgtcggacgagggcaagatgtat

SEQ ID NO: 9 - DNA, 21 bp, synthetic construct
  source 1..21: mol_type = other DNA
atgatcaacttcatccacaag

SEQ ID NO: 10 - DNA, 20 bp, synthetic construct
  source 1..20: mol_type = other DNA
tggcagtggccgagacgatc

SEQ ID NO: 11 - DNA, 20 bp, synthetic construct
  source 1..20: mol_type = other DNA
agataggtgggacgccggcg

SEQ ID NO: 12 - DNA, 19 bp, synthetic construct
  source 1..19: mol_type = other DNA
tcaaggatctcttcgaacg